SEQUENCE LISTING (16 sequences)

SEQ ID NO: 1 - DNA, 21 bp, Artificial Sequence
  CpG 1668 oligonucleotide
tccatgacgttcctgaataat

SEQ ID NO: 2 - DNA, 23 bp, Artificial Sequence
  CpG-2006
tcgtcgttttgtcgttttgtcgt

SEQ ID NO: 3 - DNA, 30 bp, Artificial Sequence
  G10 oligonucleotide
gggggggggggacgatcgtcgggggggggg

SEQ ID NO: 4 - DNA, 30 bp, Artificial Sequence; identical to SEQ ID NO: 3
  G10-PS oligonucleotide

SEQ ID NO: 5 - DNA, 30 bp, Artificial Sequence
  G6 oligonucleotide
ggggggcgacgacgatcgtcgtcggggggg

SEQ ID NO: 6 - DNA, 19 bp, Artificial Sequence
  G3-6 oligonucleotide
ggggacgatcgtcgggggg

SEQ ID NO: 7 - DNA, 20 bp, Artificial Sequence
  G4-6 oligonucleotide
gggggacgatcgtcgggggg

SEQ ID NO: 8 - DNA, 21 bp, Artificial Sequence
  G5-6 oligonucleotide
ggggggacgatcgtcgggggg

SEQ ID NO: 9 - DNA, 22 bp, Artificial Sequence
  G6-6 oligonucleotide
gggggggacgatcgtcgggggg

SEQ ID NO: 10 - DNA, 24 bp, Artificial Sequence
  G7-7 oligonucleotide
ggggggggacgatcgtcggggggg

SEQ ID NO: 11 - DNA, 26 bp, Artificial Sequence
  G8-8 oligonucleotide
gggggggggacgatcgtcgggggggg

SEQ ID NO: 12 - DNA, 28 bp, Artificial Sequence
  G9-9 oligonucleotide
ggggggggggacgatcgtcggggggggg

SEQ ID NO: 13 - protein, 9 aa, Artificial Sequence
  p33 peptide
KAVYNFATM

SEQ ID NO: 14 - DNA, 20 bp, Artificial Sequence
  1826 oligonucleotide
tccatgacgttcctgacgtt

SEQ ID NO: 15 - DNA, 21 bp, Artificial Sequence
  1668 po oligonucleotide
tccatgacgttcctgaataat

SEQ ID NO: 16 - DNA, 10 bp, Artificial Sequence
  palindromic oligonucleotide
gacgatcgtc

(19) United States
(12) Patent Application Publication
(10) Pub. No.: US 2006/0182793 A1
(43) Pub. Date: Aug. 17, 2006

(54) Cpg-packaged liposomes

(75) Inventors: Martin F. Bachmann, Seuzach (CH); Vania Manolova, Zurich (CH); Tazio Storni, Zurich (CH)

Correspondence Address: STERNE, KESSLER, GOLDSTEIN & FOX PLLC, 1100 NEW YORK AVENUE, N.W., WASHINGTON, DC, 20005, US

(73) Assignee: Cytos Biotechnology AG, Zurich-Schliern (CH)

(21) Appl. No.: 10/565,264

(22) PCT Filed: Jul. 22, 2004
(86) PCT No.: PCT/EP04/08190

§ 371 Date: Jan. 20, 2006

Related U.S. Application Data
(60) Provisional application No. 60/488,799, filed on Jul. 22, 2003.

(51) Int. Cl. A61K 48/00; A61K 9/127

(52) U.S. Cl. 424/450; 514/44

(57) ABSTRACT

Liposomes are known to enhance the activity of K- (B-) type CpGs which trigger the production of IL-12. In the present invention, the surprising finding was made that liposomes also enhance the activity of D- (A-) type CpGs, leading to the production of IFNα in vivo. These findings are relevant for the humans situation, since IFNα rather than IL-12 is the key cytokine for the induction of Th1 responses and anti-viral protection in humans.

### BACKGROUND OF THE INVENTION

1. Field of the Invention

The present invention is related to the fields of vaccinology, immunology and medicine. The invention provides compositions and methods for enhancing production of IFNα in an animal by binding or enclosing and packaging, respectively, of at least one A-type CpG, preferably oligonucleotides containing at least one non-methylated CpG sequence. Preferred liposomes are cationic liposomes. The invention can be used to induce IFNα in vivo, particularly useful for the treatment of chronic viral diseases, cancer and short-term prophylaxis from pathogen-infection.

2. Related Art

The essence of the immune system is built on two separate foundation pillars: one is specific or adaptive immunity which is characterized by relatively slow response-kinetics and the ability to remember; the other is non-specific or innate immunity exhibiting rapid response-kinetics but lacking memory. Lymphocytes are the key players of the adaptive immune system. Each lymphocyte expresses antigen-receptors of unique specificity. Upon recognizing an antigen via the receptor, lymphocytes proliferate and develop effector function. Few lymphocytes exhibit specificity for a given antigen or pathogen, and massive proliferation is usually required before an effector response can be measured—hence, the slow kinetics of the adaptive immune system. Since a significant proportion of the expanded lymphocytes survive and may maintain some effector function following elimination of the antigen, the adaptive immune system reacts faster when encountering the antigen a second time. This is the basis of its ability to remember.

In contrast to the situation with lymphocytes, where specificity for a pathogen is confined to few cells that must expand to gain function, the cells and molecules of the innate immune system are usually present in massive numbers and recognize a limited number of invariant features associated with pathogens (Medzhitov, R. and Janeway, C. A., Jr., Cell 91:295-298 (1997)). Examples of such patterns include lipopolysaccharides (LPS), non-methylated CG-rich DNA (CpG) or double stranded RNA, which are specific for bacterial and viral infections, respectively.

Most research in immunology has focused on the adaptive immune system and only recently has the innate immune system entered the focus of interest. Historically, the adaptive and innate immune system were treated and analyzed as two separate entities that had little in common. Such was the disparity that few researchers wondered why antigens were much more immunogenic for the specific immune system when applied with adjuvants that stimulated innate immunity (Sotomayor, E. M., et al., Nat. Med. 5:780 (1999); Diehl, L., et al., Nat. Med. 5:774 (1999); Weigle, W. O., Adv. Immunol. 30:159 (1980)). However, the answer posed by this question is critical to the understanding of the immune system and for comprehending the balance between protective immunity and autoimmunity.

Stimulation of innate immunity alone is able to confer non-specific protection from infection, mainly via induction of cytokines. In addition, topical and local application of stimulators of innate immunity may be able to protect from tumor growth. DNA rich in non-methylated CG motifs (CpG), as present in bacteria and most non-vertebrates, is an important example of such a stimulator of innate immunity, since CpGs exhibit a potent stimulatory activity on B cells, dendritic cells and other APC's in vitro as well as in vivo. Although bacterial DNA is immunostimulatory across many vertebrate species, the individual CpG motifs may differ. In fact, CpG motifs that stimulate mouse immune cells may not necessarily stimulate human immune cells and vice versa.

Interestingly, two types of CpGs exist, those that activate B cells and trigger the production of IL-12 (B-type, also known as K-type) and those that activate plasmocytoid DCs and induce the production of IFNα (A-type, also known as D-type). In general, B-type CpGs exhibit maximal activity only if the natural phosphodiester bond of the DNA is replaced by non-natural phosphothioester bond. This modification not only stabilizes the CpGs and protects them from degradation by nucleases but also leads to enhanced recognition by TLR9. This is different for A-type CpGs, which are optimally recognized by TLR9 in their natural phosphodiester form, while phosphothioester stabilized A-type CpGs are poorly recognized (Krieg A M, Annu Rev Immunol. 2002; 20:709-60).

Therefore, the usefulness of A-type CpGs is often limited in vivo, since they are rather unstable in vivo. Thus, they exhibit unfavourable pharmacokinetics. In order to render A-type CpG-oligonucleotides more potent, it would be essential to apply them in a protected form. One possibility to stabilize A-type CpGs is to package them into virus-like particles (VLPs), which protect them from degradation (WO03/024481). However, this leads to a concomitant strong T and B cell response against the VLPs. While this is desirable if the VLPs are used as vaccines, this is a disadvantage for non-specific stimulation of innate immunity, since it precluded multiple applications.

It has previously been shown that application of B-type CpGs in liposomes enhances their capacity to induce production of IL-12 in vitro and in vivo (J Immunol 167: 3324). However, liposomes were reported not to enhance the potency of A-type CpGs (WO 03/040308). We now found surprisingly, that liposomes strongly enhance the in vivo efficacy of A-type CpGs.

### SUMMARY OF THE INVENTION

This invention is based on the surprising finding that liposomes not only enhance the in vivo efficacy of B-type CpGs but also of A-type CpGs. This now offers the unexpected opportunity to induce high levels of IFNα in vivo using A-type CpGs.

In a first embodiment, the invention provides a composition for inducing the production of IFNα in an animal comprising a liposome and an A-type unmethylated CpG-containing oligonucleotide, where the oligonucleotide is bound to or enclosed by the liposome.

In a preferred embodiment, the at least one A-type CpG comprises at least one CpG motif, wherein all the nucleotides of the at least one CpG motif are composed of phosphodiester nucleotides. In a further preferred embodiment, the at least one A-type CpG comprises poly G motifs at the 5′ and 3′ ends, and wherein preferably all of the G nucleotides are phosphodiester nucleotides.

In a preferred embodiment of the invention, the A-type (also called D-type) CpG comprises or alternatively consists of a phosphodiester oligonucleotide, preferably comprising a palindromic sequence, wherein preferably the palindromic sequence is GACGATCGTC (SEQ ID NO: 16). In a most preferred embodiment, the A-type CpG has the sequence GGGGGGGGGGGACGATCGTCGGGGGGGGGG (SEQ ID NO: 3) or is a shorter version thereof.

In a preferred embodiment, the liposome is neutral, anionic, cationic, stealth or cationic stealth. In a most preferred embodiment, the liposome is a cationic liposome. In a further preferred embodiment the liposome is smaller than 200 mm.

In a further aspect, the present invention provides a method for enhancing the production of IFNα in an animal comprising introducing into the animal a composition of the invention.

In another aspect of the present invention, a vaccine is provided comprising an immunologically effective amount of the composition of the invention together with a pharmaceutically acceptable diluent, carrier or excipient.

The route of injection is preferably subcutaneous or intramuscular, but it would also be possible to apply the A-type CpG-containing liposomes intradermally, intranasally, intravenously or directly into the lymph node. In an equally preferred embodiment, the A-type CpG-containing liposomes mixed with antigen are applied locally, near a tumor or local viral reservoir.

It is to be understood that both the foregoing general description and the following detailed description are exemplary and explanatory only and are intended to provide further explanation of the invention as claimed.

### BRIEF DESCRIPTION OF THE DRAWINGS/FIGURES

FIG. 1 shows that phosphodiester (type A) oligonucleotides efficiently activate human CD8+ T cells from peripheral blood. Peripheral blood mononuclear cells (PBMC) were obtained from heparinized blood of healthy volunteers by ficoll (Amersham Biosciences, Sweden) density centrifugation. PBMC were resuspended in 10% FCS RPMI and plated in 96-U-bottom well plate at 0.3×10⁶ cells/well. Cells were treated with the indicated concentrations of oligonucleotides or left untreated for 24 h, at 37° C. PBMC were stained on ice with a combination of anti-CD8-FITC and anti-CD69-APC (all from Becton Dickinson, USA). Cells were acquired and analyzed using FACSCalibur (Becton Dickinson, USA).

FIG. 2 shows that phosphothioate (type B) oligonucleotides efficiently activate human B cells. Peripheral blood mononuclear cells were (PBMC) obtained from heparinized blood of healthy volunteers by ficoll (Amersham Biosciences, Sweden) density centrifugation. PBMC were resuspended in 10% FCS RPMI and plated in 96-U-bottom well plate at 0.3×10⁶ cells/well. Cells were treated with the indicated concentrations of oligonucleotides or left untreated for 24 h, at 37° C. PBMC were stained on ice with a combination of anti-CD19-PE and anti-CD69-APC (all from Becton Dickinson, USA). Cells were acquired and analyzed using FACSCalibur (Becton Dickinson, USA).

FIG. 3 shows that only phosphodiester (Type A) oligonucleotides induce IFN alpha secretion from human PBMC. Peripheral blood mononuclear cells (PBMC) were obtained from heparinized blood of healthy volunteers by ficoll (Amersham Biosciences, Sweden) density centrifugation. PBMC were resuspended in 10% FCS RPMI and plated in 96-U-bottom well plate at 0.3×10⁶ cells/well. Cells were treated with the indicated concentrations of oligonucleotides or left untreated for 24 h, at 37° C. IFN alpha, released in the supernatants was measured by ELISA using an antibody set (Cat. # 71100-1) from PBL Biomedical Laboratories, USA.

FIG. 4 shows that phosphothioester (type B) oligonucleotides induce IL-12 secretion from human PBMC. Peripheral blood mononuclear cells were (PBMC) obtained from heparinized blood of healthy volunteers by ficoll (Amersham Biosciences, Sweden) density centrifugation. PBMC were resuspended in 10% FCS RPMI and plated in 96-U-bottom well plate at 0.3×10⁶ cells/well. Cells were treated with the indicated concentrations of oligonucleotides or left untreated for 24 h, at 37° C. IL-12, released in the supernatants was measured by ELISA using an antibody pair provided from Becton Dickinson (C8.3 and C8.6 clones).

FIG. 5 shows that phosphodiester (type A) oligonucleotides induce IFN alpha secretion from human plasmacytoid DC (pDC). pDC were isolated from human PBMC by magnetic activated cell sorting (MACS). PBMC from buffy coats were labeled with anti-BDCA-2 mAb coupled to magnetic beads (Milteniy, Germany) according to manufacturer's protocol. Labeled cells were positively selected by passing PBMC through a LS column. The purity of pDC was controlled by staining them with anti-BDCA-4-APC mAb (Milteniy). pDC were plated at 0.04×10⁶/well and treated with G10, 2006 or left untreated. Twenty four hours later IFN alpha released in the supernatants was measured by ELISA, as described in the legend of FIG. 3.

FIG. 6 shows that phosphothioester-stabilized G10 (G10 PS) fails to activate human T cells. Peripheral blood mononuclear cells (PBMC) were obtained from heparinized blood of healthy volunteers by ficoll (Amersham Biosciences, Sweden) density centrifugation. PBMC were resuspended in 10% FCS RPMI and plated in 96-U-bottom well plate at 0.3×10⁶ cells/well. Cells were treated with the indicated concentrations of oligonucleotides or left untreated for 24 h, at 37° C. IFN alpha, released in the supernatants was measured by ELISA using an antibody set (Cat. # 71100-1) from PBL Biomedical Laboratories, USA.

FIG. 7 shows that 1668pt but not 1668po or G6 is able to enhance CTL responses in vivo. FIG. 7A: Mice were immunized with 100 ug of p33-VLPs (HBcAg with genetically fused the p33 epitope) alone or mixed with 1668pt or 1668po CpGs (20 nmol). Twelve days later, mice were challenged ip with recombinant vaccinia virus expressing LCMV GP (1×106 pfu) and viral titers were determined in ovaries 5 days later. FIG. 7B: The bacteriophage Qβ capsid was used as VLP, to which the p33 peptide was chemically coupled, and co-delivered with the G6 CpG. Mice were left untreated or immunized with 90 ug of Qβ p33-VLPs mixed with G6 CpGs (20 nmol). Twelve days later, mice were challenged ip with recombinant vaccinia virus expressing LCMV GP (1×10⁶ pfu) and viral titers were determined in ovaries 5 days later.

FIG. 8 shows that G6 in liposomes is able to enhance p33-specific immunity. FIG. 8A: Liposomes containing 1 mg/ml p33 peptide (KAVYNFATM) (SEQ ID NO: 13) alone or with 100 nmol/ml CpGs (1668 or G6) were produced. Subsequently, groups of C57BL/6 mice were vaccinated with the liposomal preparations (doses of 100 ug p33 peptide alone or with 10 nmol 1668 or G6 per mouse) and p33-specific T cell responses were assessed by tetramer-staining 8 days later. FIG. 8B: At day 12, liposome-treated mice were challenged ip with recombinant vaccinia virus expressing LCMV-GP (4×10⁶ pfu) and viral titers were determined in ovaries 5 days later.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

1. Definitions

Animal: As used herein, the term “animal” is meant to include, for example, humans, sheep, horses, cattle, pigs, dogs, cats, rats, mice, birds, reptiles, fish, insects and arachnids.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

The compositions and methods of the invention are also useful for treating cancer by stimulating non-specific immunity against cancer which may enhance specific immunity against tumor antigens. A “tumor antigen” as used herein is a compound, such as a peptide, associated with a tumor or cancer and which is capable of provoking an immune response. In particular, the compound is capable of provoking an immune response when presented in the context of an MHC molecule. Tumor antigens can be prepared from cancer cells either by preparing crude extracts of cancer cells, for example, as described in Cohen, et al., Cancer Research, 54:1055 (1994), by partially purifying the antigens, by recombinant technology or by de novo synthesis of known antigens. Tumor antigens include antigens that are antigenic portions of or are a whole tumor or cancer polypeptide. Such antigens can be isolated or prepared recombinantly or by any other means known in the art. Cancers or tumors include, but are not limited to, biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas.

Allergens also serve as antigens in vertebrate animals. The term “allergen”, as used herein, also encompasses “allergen extracts” and “allergenic epitopes.” Examples of allergens include, but are not limited to: pollens (e.g. grass, ragweed, birch and mountain cedar); house dust and dust mites; mammalian epidermal allergens and animal danders; mold and fungus; insect bodies and insect venom; feathers; food; and drugs (e.g., penicillin).

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Antigen presenting cell: As used herein, the term “antigen presenting cell” is meant to refer to a heterogeneous population of leucocytes or bone marrow derived cells which possess an immunostimulatory capacity. For example, these cells are capable of generating peptides bound to MHC molecules that can be recognized by T cells. The term is synonymous with the term “accessory cell” and includes, for example, Langerhans' cells, interdigitating cells, dendritic cells, B cells and macrophages. Under some conditions, epithelial cells, endothelial cells and other, non-bone marrow derived cells may also serve as antigen presenting cells.

Bound: As used herein, the term “bound” refers to binding that may be covalent, e.g., by chemically coupling the unmethylated CpG-containing oligonucleotide to a liposome, or non-covalent, e.g., ionic interactions, hydrophobic interactions, hydrogen bonds, etc. Covalent bonds can be, for example, ester, ether, phosphoester, amide, peptide, imide, carbon-sulfur bonds, carbon-phosphorus bonds, and the like. The term also includes the enclosement, or partial enclosement, of a substance. The term “bound” is broader than and includes terms such as “coupled,” “fused,” “enclosed” and “attached.” Moreover, with respect to the CpG being bound to the liposome, the term “bound” also includes the enclosement, or partial enclosement, of the CpG. Therefore, with respect to the CpG being bound to the liposome the term “bound” is broader than and includes terms such as “coupled,” “fused,” “enclosed”, “packaged” and “attached.” For example, the CpG can be enclosed by the liposome without the existence of an actual binding, neither covalently nor non-covalently, such that the oligonucleotide is held in place by mere “packaging.”

CpG: As used herein, the term “CpG” refers to an oligonucleotide which contains at least one unmethylated cytosine, guanine dinucleotide sequence (e.g. “CpG-oligonucleotides” or DNA containing a cytosine followed by guanosine and linked by a phosphate bond) and stimulates/activates, e.g. has a mitogenic effect on, or induces or increases cytokine expression by, a vertebrate bone marrow derived cell. Preferably, as used herein, a CpG oligonucleotide is an oligonucleotide that is at least about ten nucleotides in length and includes at least one unmethylated CpG dinucleotide. The entire CpG oligodeoxynucleotide can be unmethylated or portions may be unmethylated. For example, CpGs can be useful in activating B cells, NK cells and antigen-presenting cells, such as dendritic cells, monocytes and macrophages. The CpGs can include nucleotide analogs such as analogs containing phosphorothioester bonds and can be double-stranded or single-stranded. Generally, phosphothioester stabilized CpGs are B-type CpGs while phosphodiester CpGs are A-type CpGs as indicated below.

“CpG motif”: As used herein, the term “CpG motif” refers to a pattern of nucleotides that include an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5′ and 3′ ends. Without being bound by theory, the bases flanking the CpG confer a significant part of the activity to the CpG oligonucleotide.

A-type CpGs: As used herein, the term “A-type CpG” or “D-type CpG” refers to an oligodeoxynucleotide (ODN) comprising at least one CpG motif. The nucleotides of the at least one CpG motif are linked by at least one, typically and preferably exclusively phosphodiester (PO) bonds. Preferably, the CpG motif, and hereby preferably the CpG dinucleotide and its immediate flanking regions comprising at least one, preferably two nucleotides, are composed of phosphodiester nucleotides. Typically and preferably, the term “A-type CpG” or “D-type CpG” as used within this specification, refers to an oligodeoxynucleotide (ODN) comprising at least one CpG motif and having poly G motifs at the 5′ and/or 3′ ends. Typically and preferably, the poly G motif comprises or alternatively consists of at least one, preferably at least three, at least four, at least five, at least six, at least seven, at least 8, at least 9, and more preferably at least 10 Gs (glycins). In some embodiments, the 5′ and/or 3′ ends, typically and preferably at least one G of the poly G motifs at the 5′ and/or 3′ ends, preferably at least two, three or four, even more preferably all Gs of the poly G motif, are phoshorothioate modified. In a very preferred embodiment, all Gs of the poly G motif are linked by phosphodiester bonds. A-type CpGs preferentially stimulate activation of T cells and the maturation of dendritic cells and induce the release of IFNα. Preferably, the A-type CpG of the invention comprises or alternatively consists of a palindromic sequence. Typically and preferably, the CpG motif is part of a palindromic sequence. Typically and preferably, all nucleotides, preferably at least the CpG motif of the palindromic sequence, are composed of phosphodiester nucleotides. Typically and preferably, the palindromic sequence is GACGATCGTC (SEQ ID NO: 16).

Immune response: As used herein, the term “immune response” refers to the systemic or local production of cytokines/chemokines/interferons. In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent.

Immunization: As used herein, the terms “immunize” or “immunization” or related terms refer to conferring the ability to mount a substantial immune response (including non-specific production of cytokines, chemokines, interferons and alike). These terms do not require that complete immunity be created, but rather that an immune response be produced which is substantially greater than baseline. For example, a mammal may be considered to be immunized if systemic or local cytokine/chemokine/interferon production can be measured.

Liposome: As used herein, the term “liposome” refers to phospholipid vesicles comprising one or more, preferably one, two, or three phospholipid bilayer membranes. Liposomes vary in charge and in size depending on the method of preparation and the lipids used. The liposome of the present invention may be neutral, cationic, anionic, stealth, or cationic stealth. Preferably, the liposome of the invention is a cationic liposome. The liposome may have a diameter between 100 and 800 nm, preferably between 100 and 400 nm, more preferably between 100 and 300 nm, even more preferably between 100 and 200 nm, most preferably less than 200 nm. The term “liposome”, as used herein, shall also encompass modified liposomes, preferably modified liposomes, wherein the surface of the liposomes may be specifically modified to optimize binding to DC, for example, via specific sugar moieties (Fukasawa et al., (1998), FEBS, 441, 353-356) or antibodies (Serre et al. (1998), J. Immunol., 161, 6059-6067).

Oligonucleotide: As used herein, the terms “oligonucleotide” or “oligomer” refer to a nucleic acid sequence comprising 2 or more nucleotides, generally at least about 6 nucleotides to about 100,000 nucleotides, preferably about 6 to about 2000 nucleotides, and more preferably about 6 to about 300 nucleotides, even more preferably about 20 to about 300 nucleotides, and even more preferably about 20 to about 100 nucleotides. The terms “oligonucleotide” or “oligomer” also refer to a nucleic acid sequence comprising more than 100 to about 2000 nucleotides, preferably more than 100 to about 1000 nucleotides, and more preferably more than 100 to about 500 nucleotides. “Oligonucleotide” also generally refers to any polyribonucleotide or polydeoxribonucleotide, which may be unmodified RNA or DNA or modified RNA or DNA. The modification may comprise the backbone or nucleotide analogues. “Oligonucleotide” includes, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “oligonucleotide” refers to triple-stranded regions comprising RNA or DNA or both RNA and DNA. Further, an oligonucleotide can be synthetic, genomic or recombinant, e.g., λ-DNA, cosmid DNA, artificial bacterial chromosome, yeast artificial chromosome and filamentous phage such as M13.

The term “oligonucleotide” also includes DNAs or RNAs containing one or more modified bases and DNAs or RNAs with backbones modified for stability or for other reasons. For example, suitable nucleotide modifications/analogs include peptide nucleic acid, inosin, tritylated bases, phosphorothioates, alkylphosphorothioates, 5-nitroindole deoxyribofuranosyl, 5-methyldeoxycytosine and 5,6-dihydro-5,6-dihydroxydeoxythymidine. A variety of modifications have been made to DNA and RNA; thus, “oligonucleotide” embraces chemically, enzymatically or metabolically modified forms of polynucleotides as typically found in nature, as well as the chemical forms of DNA and RNA characteristic of viruses and cells. Other nucleotide analogs/modifications will be evident to those skilled in the art.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the production of cytokines and alike. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

The compositions of the invention can be combined, optionally, with a pharmaceutically-acceptable carrier. The term “pharmaceutically-acceptable carrier” as used herein means one or more compatible solid or liquid fillers, diluents or encapsulating substances which are suitable for administration into a human or other animal. The term “carrier” denotes an organic or inorganic ingredient, natural or synthetic, with which the active ingredient is combined to facilitate the application.

Treatment: As used herein, the terms “treatment”, “treat”, “treated” or “treating” refer to prophylaxis and/or therapy. When used with respect to an infectious disease, for example, the term refers to a prophylactic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse.

Vaccine: As used herein, the term “vaccine” refers to a formulation which contains the composition of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

Optionally, the vaccine of the present invention additionally includes an adjuvant which can be present in either a minor or major proportion relative to the compound of the present invention. The term “adjuvant” as used herein refers to non-specific stimulators of the immune response or substances that allow generation of a depot in the host which when combined with the vaccine of the present invention provide for an even more enhanced immune response. A variety of adjuvants can be used. Examples include incomplete Freund's adjuvant, aluminum hydroxide and modified muramyldipeptide.

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd. edition, Cold Spring Harbor Laboratory Press, Cold Spring Harbor, N.Y. (1989).

2. Compositions and Methods for Enhancing of CpG-Induced INFα-Production by Liposomes:

The disclosed invention provides compositions and methods for enhancing the production of IFNα by CpGs in an animal. Compositions of the invention comprise, or alternatively consist of, (a) a liposome and (b) at least one A-type CpG, wherein said A-type CpG (b) is bound to or enclosed by the liposome (a). Preferably, the A-type CpG of the invention is G10 (SEQ ID NO: 3). Furthermore, the invention provides a method for enhancing the production of IFNα in an animal comprising introducing into said animal a composition of the invention. In a further aspect, the invention provides a method of immunizing or treating an animal comprising administering to the animal an immunologically effective amount of a vaccine of the invention. In addition, the invention conveniently enables the practitioner to construct such a composition for various treatment and/or prevention purposes, which include the prevention and/or treatment of infectious diseases, as well as chronic infectious diseases, the prevention and/or treatment of cancers.

In preferred embodiment of the invention, the A-type CpG comprises or consists of a CpG motif with bases linked by phosphodiester bonds. In a further embodiment, the at least one A-type CpG of the invention comprises poly G motifs at the 5′ and 3′ ends, preferably wherein the G bases are phosphodiester bases. In some embodiments, the 5′ and 3′ ends, typically and preferably the poly G motifs at the 5′ and 3′ ends, are phoshorothioate modified. In a very preferred embodiment, the CpG motif is part of a palindromic sequence.

In some embodiments, the A-type CpG oligonucleotide comprises or consists of an unmethylated CpG motif that has a sequence represented by the formula: 5′R₁Y₁—CG-R₂Y₂3′, wherein the central CpG motif is unmethylated, and R₁, R₂, Y₁, and Y₂ are any nucleotide. In other embodiments, the unmethylated CpG motif has a sequence represented by the formula:

5′R₁Y₁CGR₂Y₂ 3′, 5′R₁Y₁CGY₂R₂3′, 5′R₁R₂CGR₃Y₁3′, R₃Y₁CGY₂Y₃ 3′ or preferably 5′R₁R₂CGR₃Y₁CGY₂Y₃ 3′, wherein the CpG motif is unmethylated, and wherein R₁, R₂, or R₃ is A or G (a purine), and Y₁, Y₂, or Y₃ is C or T (a pyrimidine). In one embodiment, an A-type CpG is at least about 16 nucleotides in length and comprises or contains a sequence represented by formula: 5′-(G)_{K}(X)_{L}RYCGYR(W)_{M}(G)_{N}-3′ wherein the central CpG motif is unmethylated, R is a purine nucleotide, Y is a pyrimidine nucleotide, X and W are any nucleotide, K is any integer from 3 to 10, L is any integer from 0 to 10, M is any integer from 0 to 10, and N is any integer from 4 to 10.

In addition, the oligonucleotide can comprise about 6 to about 100,000 nucleotides, preferably about 6 to about 2000 nucleotides, more preferably about 20 to about 2000 nucleotides, more preferably about 20 to about 300 nucleotides, more preferably about 20 to about 100 nucleotides, and even more preferably about 20 to about 40 nucleotides. In addition, the oligonucleotide can comprise more than 100 to about 2000 nucleotides, preferably more than 100 to about 1000 nucleotides, and more preferably more than 100 to about 500 nucleotides.

In one embodiment, the A-type CpG-containing oligonucleotide contains one or more phosphothioester modifications of the phosphate backbone. For example, an A-type CpG-containing oligonucleotide having one or more phosphate backbone modifications or preferably, having the phosphate backbone of the poly G motif modified, wherein one, some or all of the nucleotide phosphate backbone modifications are phosphorothioate modifications are included within the scope of the present invention. In one embodiment, the poly G motif at the 5′ and 3′ ends of the A-type CpG oligonucleotide, contains phosphorohioate modifications, and the CpG motif contains phosphodiester nucleotides. In a preferred embodiment, all nucleotides of the A-type CpG oligonucleotide are phosphodiester nucleotides.

The at least one unmethylated A-type CpG-containing oligonucleotide can also be recombinant, genomic, synthetic, cDNA, plasmid-derived and single or double stranded. For use in the instant invention, the nucleic acids can be synthesized de novo using any of a number of procedures well known in the art, for example, the b-cyanoethyl phosphoramidite method (Beaucage, S. L., and Caruthers, M. H., Tet. Let. 22:1859 (1981); nucleoside H-phosphonate method (Garegg et al., Tet. Let. 27:4051-4054 (1986); Froehler et al., Nucl. Acid. Res. 14:5399-5407 (1986); Garegg et al., Tet. Let. 27:4055-4058 (1986), Gaffney et al., Tet. Let. 29:2619-2622 (1988)). These chemistries can be performed by a variety of automated oligonucleotide synthesizers available in the market. Alternatively, CpGs can be produced on a large scale in plasmids, (see Sambrook, T., et al., “Molecular Cloning: A Laboratory Manual,” Cold Spring Harbor laboratory Press, New York, 1989) which after being administered to a subject are degraded into oligonucleotides. Oligonucleotides can be prepared from existing nucleic acid sequences (e.g., genomic or cDNA) using known techniques, such as those employing restriction enzymes, exonucleases or endonucleases.

In another preferred embodiment of the present invention, the CpG motif of said at least one unmethylated A-type CpG-containing oligonucleotide is part of a palindromic sequence. Preferably said palindromic sequence is GACGATCGTC (SEQ ID NO: 16). In another preferred embodiment, the palindromic sequence is flanked at its 3′-terminus and at its 5′-terminus by 10 guanosine entities, wherein preferably said palindromic sequence is GACGATCGTC (SEQ ID NO: 16). In another embodiment, said palindromic sequence is GACGATCGTC (SEQ ID NO: 16), and wherein said palindromic sequence is flanked at its 3′-terminus and at its 5′-terminus by more than two and less than 11 guanosine entities or, more preferably by 8-10 guanosine entities, or, most preferably by 10 guanosine entities.

In a preferred embodiment of the present invention, the palindromic sequence comprises, or alternatively consist essentially of, or alternatively consists of or is GACGATCGTC (SEQ ID NO: 16), and the palindromic sequence is flanked at its 5′-terminus by at least 3 and at most 10 guanosine entities and wherein said palindromic sequence is flanked at its 3′-terminus by at least 6 and at most 10 guanosine entities. In another embodiment, the palindromic sequence is flanked at its 5′-terminus by at least 3 and at most 10 guanosine entities and wherein said palindromic sequence is flanked at its 3′-terminus by at least 6 and at most 10 guanosine entities.

In a further very preferred embodiment of the present invention, the at least one unmethylated A-type CpG-containing oligonucleotide comprises, or alternatively consists essentially of, or alternatively consists of a palindromic sequence, wherein at least one unmethylated A-type CpG-containing oligonucleotide comprises or consists of a nucleic acid sequence selected from the group consisting of (a) GGGGACGATCGTCGGGGGG ((SEQ ID NO: 6); and typically abbreviated herein as G3-6), (b) GGGGGACGATCGTCGGGGGG ((SEQ ID NO: 7); and typically abbreviated herein as G4-6), (c) GGGGGGACGATCGTCGGGGGG ((SEQ ID NO: 8); and typically abbreviated herein as G5-6), (d) GGGGGGGACGATCGTCGGGGGG ((SEQ ID NO: 9); and typically abbreviated herein as G6-6), (e) GGGGGGGGACGATCGTCGGGGGGG ((SEQ ID NO: 10); and typically abbreviated herein as G7-7), (f) GGGGGGGGGACGATCGTCGGGGGGGG ((SEQ ID NO: 11); and typically abbreviated herein as G8-8), (g) GGGGGGGGGGACGATCGTCGGGGGGGGG ((SEQ ID NO: 12); and typically abbreviated herein as G9-9), (h) GGGGGGCGACGACGATCGTCGTCGGGGGGG ((SEQ ID NO: 5); and typically abbreviated herein as G6), and (i) GGGGGGGGGG GACGATCGTCGGGGGGGGGG ((SEQ ID NO: 3) and typically abbreviated herein as G10).

In a further preferred embodiment of the present invention the CpG motif of the at least one unmethylated A-type CpG-containing oligonucleotide is part of a palindromic sequence, wherein said palindromic sequence is GACGATCGTC (SEQ ID NO: 16), and wherein said palindromic sequence is flanked at its 5′-terminus of at least 4 and at most 9 guanosine entities and wherein said palindromic sequence is flanked at its 3′-terminus of at least 6 and at most 9 guanosine entities.

In a further preferred embodiment of the present invention the CpG motif of the at least one unmethylated A-type CpG-containing oligonucleotide is part of a palindromic sequence, wherein said palindromic sequence is GACGATCGTC (SEQ ID NO: 16), and wherein said palindromic sequence is flanked at its 5′-terminus of at least 5 and at most 8 guanosine entities and wherein said palindromic sequence is flanked at its 3′-terminus of at least 6 and at most 8 guanosine entities.

Liposomes in the context of the present application refer to lipid vesicles consisting of a lipid bilayer that can be used to entrap or bind various drugs including CpGs. The liposome of the present invention may be selected from the group consisting of neutral liposome, anionic liposome, cationic liposome, stealth, or cationic stealth. In a preferred embodiment, the liposome is a cationic liposome. The liposome may have a diameter between 100 and 800 nm, preferably between 100 and 400 nm, more preferably between 100 and 300 nm, even more preferably between 100 and 200 nm, most preferably 200 nm.

In a preferred embodiment, the liposome exhibits positive charges in order to facilitate interaction of T cells with target cells. In some embodiments, the liposome comprises a cationic lipid, a colipid, and a stabilizing additive. In another embodiment, the liposome comprises dimethylaminoethane-carbamol-cholisterol, and/or dioleoylphosphatidylethanolamine, and/or polyethylene glycol derivatized phosphatidylethanolamine. In a preferred embodiment, the liposome comprises phosphatidylcholine, and/or cholesterol, and/or DL-α-tocopherol, preferably phosphatidylcholine, cholesterol, and DL-α-tocopherol. Generation of such liposomes is well established e.g. in Bangham et al., (1965), J. Mol. Biol., 13, 238-252; Gursel et al., (2001), J Immunol 167: 3324; or Ludewig et al., (2000), Vaccine, 19, 23-32, the disclosure of which is incorporated herein by reference in its entirety.

In one aspect of the invention, the A-type CpGs in liposomes are used to induce systemically increased levels of IFNα. Such elevated levels of IFNa are known to be therapeutically active during hepatitis B and hepatitis C virus infection and also during infection with HIV. Moreover, IFNa non-specifically protects from viral and some bacterial infection, rendering A-type CpGs in liposomes ideal prophylactic “non-specific” vaccines against infections in general. In addition, local application of A-type CpGs, as eg injection into tumors, has been shown to protect from tumor growth. Thus, A-type CpGs in liposomes may be particularly attractive for the treatment of cancer.

Therefore, in a further aspect, the invention provides a method for enhancing the production of IFNα in an animal comprising introducing into said animal a composition of the invention.

The invention also provides vaccine compositions which can be used for preventing and/or attenuating diseases or conditions. Vaccine compositions of the invention comprise, or alternatively consist of, an immunologically effective amount of the inventive immune enhancing composition together with a pharmaceutically acceptable diluent, carrier or excipient. The vaccine can also optionally comprise an adjuvant. In a preferred embodiment, the vaccine does not comprise an antigen.

In yet another aspect, the invention provides a method of immunizing an animal or treating a disease or condition in an animal, the method comprising administering to the animal an immunologically effective amount of a composition or vaccine of the invention, wherein the disease or condition is selected from the group consisting of infectious disease (e.g. virus or parasitic infections) and cancer.

The invention further provides vaccination methods for preventing and/or attenuating diseases or conditions in animals. In one embodiment, the invention provides vaccines for the prevention of infectious diseases in a wide range of animal species, particularly mammalian species such as human, monkey, cow, dog, cat, horse, pig, etc. Vaccines can be designed to treat infections of viral etiology such as HIV, influenza, Herpes, viral hepatitis, Epstein Barr, polio, viral encephalitis, measles, chicken pox, etc.; or infections of bacterial etiology such as pneumonia, tuberculosis, syphilis, etc.; or infections of parasitic etiology such as malaria, trypanosomiasis, leishmaniasis, trichomoniasis, amoebiasis, etc.

In another embodiment, the invention provides vaccines for the prevention of cancer in a wide range of species, particularly mammalian species such as human, monkey, cow, dog, cat, horse, pig, etc. Vaccines can be designed to treat all types of cancer including, but not limited to, lymphomas, carcinomas, sarcomas and melanomas.

In a further aspect, the present invention provides the use of a composition or a vaccine of the invention in the manufacture of a pharmaceutical for the treatment of a disorder or disease, wherein the disease or disorder is typically and preferably selected from the group consisting of cancer and infectious diseases.

As would be understood by one of ordinary skill in the art, when compositions of the invention are administered to an animal, they can be in a composition which contains salts, buffers, adjuvants or other substances which are desirable for improving the efficacy of the composition. Examples of materials suitable for use in preparing pharmaceutical compositions are provided in numerous sources including Remington's Pharmaceutical Sciences (Osol, A, ed., Mack Publishing Co., (1990)).

The compositions of the present invention can be administered by various methods known in the art. The particular mode selected will depend of course, upon the particular composition selected, the severity of the condition being treated and the dosage required for therapeutic efficacy. The methods of the invention, generally speaking, can be practiced using any mode of administration that is medically acceptable, meaning any mode that produces effective levels of the active compounds without causing clinically unacceptable adverse effects. Such modes of administration include oral, rectal, parenteral, intracistemal, intravaginal, intraperitoneal, topical (as by powders, ointments, drops or transdermal patch), bucal, or as an oral or nasal spray. The term “parenteral” as used herein refers to modes of administration which include intravenous, intramuscular, intraperitoneal, intrasternal, subcutaneous and intraarticular injection and infusion. The composition of the invention can also be injected directly in a lymph node.

Dosage levels depend on the mode of administration, the nature of the subject, and the quality of the carrier/adjuvant formulation. Typical amounts are in the range of about 0.1 μg to about 100 mg CpG per subject. Preferred amounts are at least about 10 μg to about 1000 μg per subject. Multiple administration to immunize the subject is preferred, and protocols are those standard in the art adapted to the subject in question.

The compositions can conveniently be presented in unit dosage form and can be prepared by any of the methods well-known in the art of pharmacy. Methods include the step of bringing the compositions of the invention into association with a carrier which constitutes one or more accessory ingredients. In general, the compositions are prepared by uniformly and intimately bringing the compositions of the invention into association with a liquid carrier, a finely divided solid carrier, or both, and then, if necessary, shaping the product.

Compositions suitable for oral administration can be presented as discrete units, such as capsules, tablets or lozenges, each containing a predetermined amount of the compositions of the invention. Other compositions include suspensions in aqueous liquids or non-aqueous liquids such as a syrup, an elixir or an emulsion.

Other delivery systems can include time-release, delayed release or sustained release delivery systems. Such systems can avoid repeated administrations of the compositions of the invention described above, increasing convenience to the subject and the physician. Many types of release delivery systems are available and known to those of ordinary skill in the art.

Other embodiments of the invention include processes for the production of the compositions of the invention and methods of medical treatment for cancer and allergies using said compositions.

The following examples are illustrative only and are not intended to limit the scope of the invention as defined by the appended claims. It will be apparent to those skilled in the art that various modifications and variations can be made in the methods of the present invention without departing from the spirit and scope of the invention. Thus, it is intended that the present invention cover the modifications and variations of this invention provided they come within the scope of the appended claims and their equivalents.

All patents, patent applications and publications referred to herein are expressly incorporated by reference in their entirety.

Table 1: Terminology and sequences of CpG oligonucleotides used throughout the specification.

Small letters indicate deoxynucleotides connected via phosphorothioate bonds while large letters indicate deoxynucleotides connected via phosphodiester bonds

| | | |
|---|---|---|
| | | SEQ ID |
| Terminology | Sequence | NO |

| | | |
|---|---|---|
| 1668 (1668pt) | tccatgacgttcctgaataat | 1 |
| 1668po | TCCATGACGTTCCTGAATAAT | 15 |
| 2006 | tcgtcgttttgtcgttttgtcgt | 2 |
| G10 (G10-PO) | GGGGGGGGGGGACGATCGTCGGGGGGGGGG | 3 |
| G10-PS | gggggggggggacgatcgtcgggggggggg | 4 |
| G6 | GGGGGGCGACGACGATCGTCGTCGGGGGGG | 5 |
| G3-6 | GGGGACGATCGTCGGGGGG | 6 |
| G4-6 | GGGGGACGATCGTCGGGGGG | 7 |
| G5-6 | GGGGGGACGATCGTCGGGGGG | 8 |
| G6-6 | GGGGGGGACGATCGTCGGGGGG | 9 |
| G7-7 | GGGGGGGGACGATCGTCGGGGGGG | 10 |
| G8-8 | GGGGGGGGGACGATCGTCGGGGGGGG | 11 |
| G9-9 | GGGGGGGGGGACGATCGTCGGGGGGGGG | 12 |
| 1826 | tccatgacgttcctgacgtt | 14 |

### EXAMPLES

### Example 1

G10 and Analogues Activate T Cells in Human Blood Cultures More Efficiently than CpG 2006

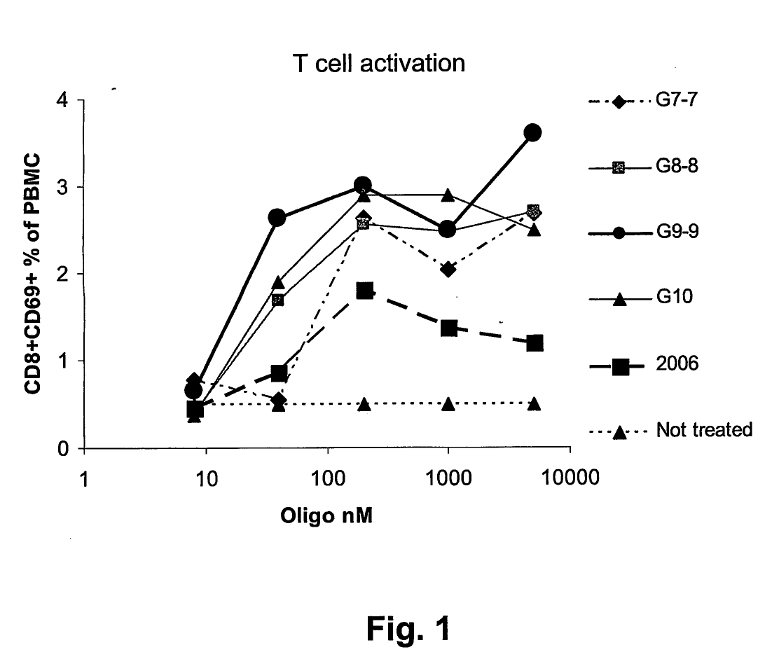

Human peripheral blood mononuclear cells (PBMC) were isolated and stimulated with various concentrations of CpG G10, G9-9, G8-8, G7-7 or the thioester stabilized CpG 2006. The next day, cells were stained for the expression of CD8 and CD69 in order to test for T cell activation. G10, G9-9, G8-8, G7-7 all efficiently activated CD8+ T cells, with G10 and G9-9 being most effective while G7-7 was least effective. In contrast, 2006 was barely able to activate human T cells (FIG. 1). This characterizes G10, G9-9, G8-8, G7-7 as A type CpGs while 2006 is characterized as a B type CpG.

### Example 2

2006 but not G10 and Analogues Activate B Cells in Human Blood Cultures

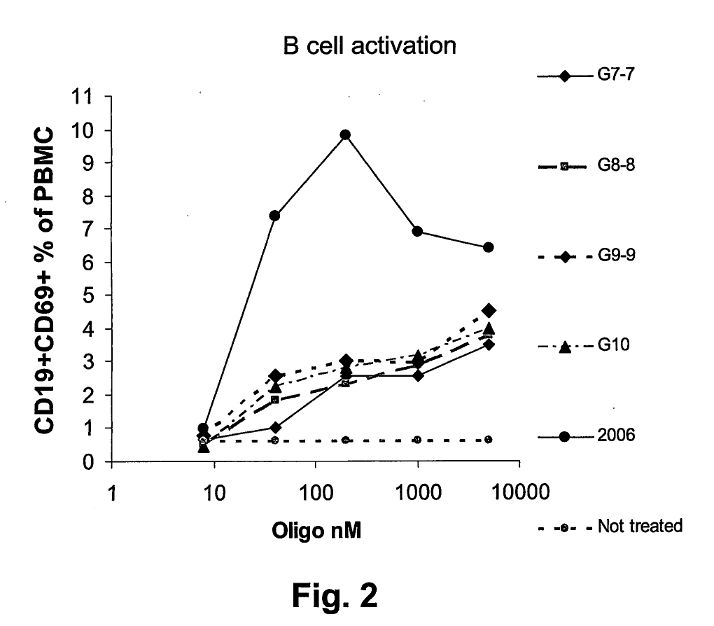

Human PBMC were isolated and stimulated with various concentrations of CpG G10, G9-9, G8-8, G7-7 or the thioester stabilized CpG 2006. The next day, cells were stained for the expression of CD19 and CD69 in order to test for B cell activation. G10, G9-9, G8-8, G7-7 failed to efficiently activate B cells. In contrast, 2006 was very effective at activating human B cells (FIG. 2). This characterizes G10, G9-9, G8-8, G7-7 as A type CpGs while 2006 is characterized as a B type CpG.

### Example 3

G10 and Analogues but not CpG 2006 Induce Production of IFNα in Human PBMC

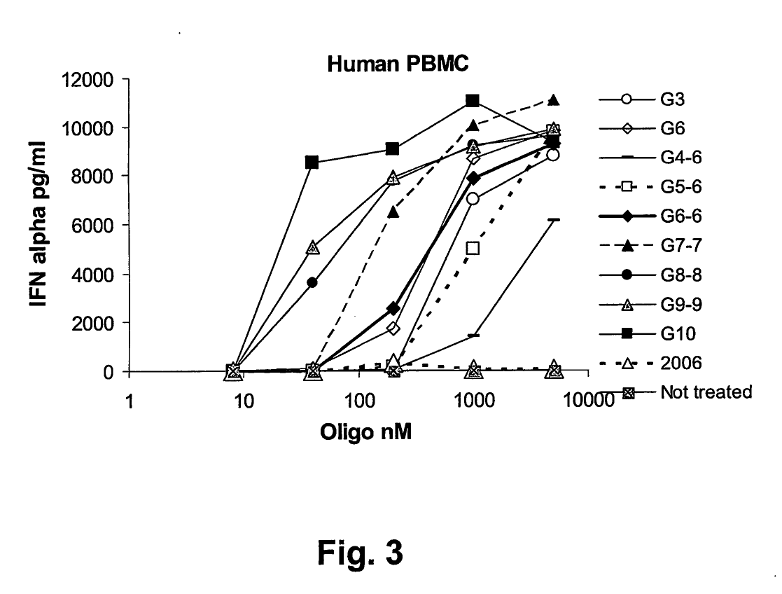

Human PBMC were isolated and stimulated with various concentrations of CpG G10, G9-9, G8-8, G7-7, G3, G6, G4-6 and G6-6 or the thioester stabilized CpG 2006. 24 h later, supernatants were assessed for the presence of IFNα by ELISA. G10, G9-9, G8-8, G7-7, G3, G6, G4-6 and G6-6 all efficiently induced the production of IFNα, with G10 being most effective while G4-6 least effective. In contrast, 2006 was not able to induce IFN alpha release from human PBMC (FIG. 3). This characterizes G10, G9-9, G8-8, G7-7 as A type CpGs while 2006 is characterized as a B type CpG.

### Example 4

2006 and 1668 but not G10 Induce Production of IL-12 in Human Blood Cultures

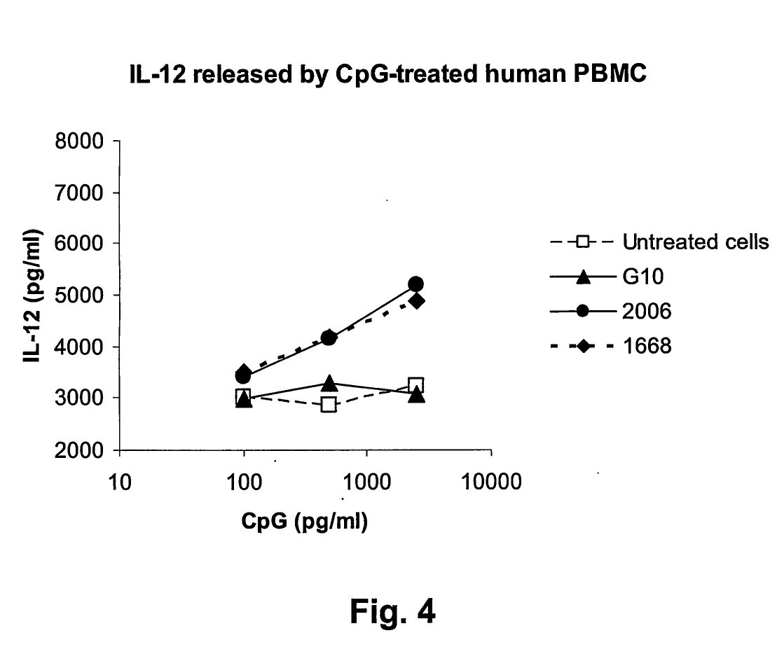

Human blood cells were isolated stimulated with various concentrations of CpG G10 or the thioester stabilized CpG 2006 or 1668. 24 h later, presence of IL-12 was assessed in the supernatant by ELISA. G10 failed to induce production of IL-12 while both thioesterstabilzed CpGs efficiently triggered the release of IL-12 (FIG. 4). This characterizes G10 as A type CpGs while 2006 and 1668 are characterized as a B type CpG.

### Example 5

G10 but not 2006 Induces Production of IFNα in Human Plasmocytoid DCs

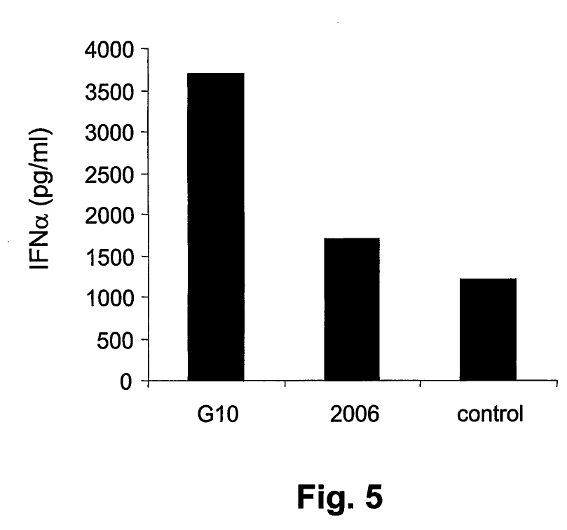

Human plasmocytoid dendritic cells (pDCs) were isolated from PBMC by labeling them with anti-BDCA-2 mAb attached to magnetic beads (Miltenyi Biotec, Germany). pDCs were subsequently stimulated with the CpGs G10 or the phosphothioester stabilized CpG 2006 (20 nM) and release of IFNα into the supernatant was monitored subsequently by ELISA. Only G10 but not 2006 was able to efficiently trigger release of IFNα (FIG. 5).

### Example 6

Phosphothioester Stabilized G10 (G10-PS) Fails to Stimulate T Cells in Human Blood Cultures

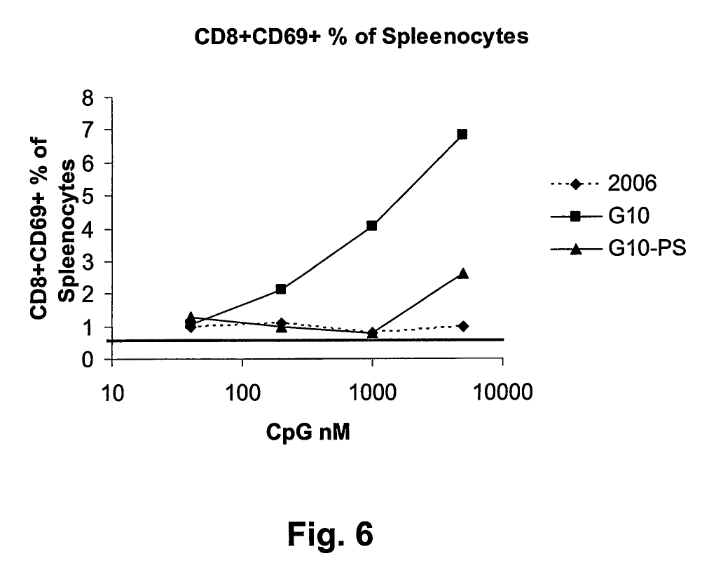

Human blood cells were isolated and stimulated with various concentrations of CpG G10 (G10-PO) or the thioester stabilized CpG G10 (G10-PS). 24 h later IFN alpha released in the supernatants was measured by ELISA. G10 efficiently induced production of IFN alpha, while the thioester stabilized version was barely active. 2006 failed to induce IFN alpha secretion (FIG. 6). Thus, thioester-stabilzed G10 (G10-PS) does not behave as an A-type CpG (e.g. G10-PO).

### Example 7

1668pt but not 1668po or G6 is Able to Enhance CTL Responses In Vivo

CpGs are able to non-specifically activate antigen-presenting cells. However, in vivo, usually only thioester-stabilized oligonucleotides may be active. We have previously observed that thioester stabilized CpGs are able to enhance CTL responses in vivo if mixed together with VLPs (Storni et al. (2002), J. Immunol. 168: 2880). We now compared the ability of 1668pt (B type, phosphorothioate stabilized 1668) CpGs with 1668po (A-type) CpGs to enhance CTL responses upon mixing with VLPs. As a model VLP, hepatitis B core Ag fused to peptide p33 derived from LCMV was used (see WO 03/024481, Example 1). The p33-VLPs were generated as follows: Hepatitis B clone pEco63 containing the complete viral genome of Hepatitis B virus was purchased from ATCC. The gene encoding HBcAg was introduced into the EcoRI/HindIII restriction sites of expression vector pKK223.3 (Amersham Pharmacia Biotech Inc., NJ) under the control of a tac promotor. The p33 peptide (KAVYNFATM, SEQ ID NO: 13)) derived from LCMV was fused to the C-terminus of HBcAg (aa 1-183) via a three leucine-linker by standard PCR methods. E. coli K802d were transfected with the plasmid and grown in 2 liter cultures until an optical density of 1 (at 600 nm wavelength). Cells were induced by adding IPTG (Sigma, Division of Fluka AG, Switzerland) to a final concentration of 1 mM for 4 hours. Bacteria were then collected by centrifugation and resuspended in 5 ml lysis buffer (10 mM Na₂HPO₄, 30 mM NaCl, 10 mM EDTA, 0.25% Tween-20, pH 7.0). 200 μl of lysozyme solution (20 mg/ml) was added. After sonication 4 μl benzonase (Merck, Darmstadt, Germany) and 10 mM MgCl₂ were supplemented to the cell lysate. The suspension was then incubated for 30 minutes at RT and centrifuged for 15 minutes at 27000×g. The retained supernatant was complemented with 20% (w/v) ammonium sulfate. After incubation for 30 minutes on ice and centrifugation for 15 minutes at 48000×g the supernatant was discarded and the pellet resuspended in 2-3 ml phosphate-saline buffer. The preparation was loaded onto a Sephacryl S-400 gel filtration column (Amersham Pharmacia Biotech Inc., NJ) for purification. Fractions were analyzed for protein content in a SDS PAGE gel and samples containing pure HBc capsids were pooled.

Electron microscopy was performed according to standard protocols.

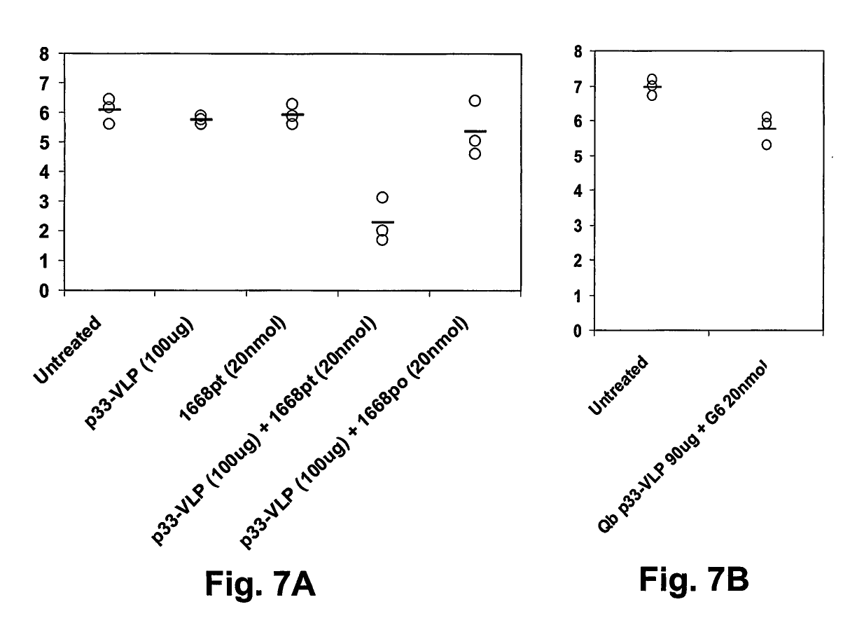

Mice were immunized with 100 μg of p33-VLPs alone or mixed with 1668pt or 1668po CpGs (20 nmol). Twelve days later, mice were challenged ip (intraperitoneal) with recombinant vaccinia virus expressing LCMV GP (1×10⁶ pfu, plaque forming unit) and viral titers were determined in ovaries 5 days later (Storni et al., 2002, J. Immunol. 168: 2880) (FIG. 7 A). Only 1668pt but not 1668po was able to enhance protective p33-specific CTL responses.

Alternatively, the bacteriophage Qβ capsid was used as VLP and co-delivered with the G6 CpG (FIG. 7 B). Production and purification of Qβ is performed with the same protocol as for HBcAg VLPs. The p33 peptide was chemically coupled to the Qβ VLP via a bifunctional linker as follows: purified QβVLPs (1.5 mg/ml in 20 mM HEPES, 150 mM NaCl pH 7.2) were derivatized by a 30 min incubation at RT with a 10-fold molar excess of succinimidyl-6-(β-maleimidopropionamido)hexanoate (Pierce Biotechnology, Rockford, Ill., USA). Free cross-linker was removed by extensive dialysis against 20 mM HEPES pH 7.2. Peptide p33 was produced in a modified version with three additional amino acids (GGC) added to the C-terminus (p33-GGC) (EMC microcollections GmbH, Tübingen, Germany) to allow coupling to VLPs. Derivatized Qβ VLPs and p33-GGC (peptide at 5-fold molar excess) were then incubated for 2 h at RT to allow cross-linking. Free p33-GGC was removed by dialysis against 20 mM HEPES pH 7.2 using DispoDialyser membranes with a molecular weight cut-off of 300 kD (Spectrum Medical Industries Inc., Rancho Dominguez, Calif.). Efficiency of cross-linking was analysed by SDS polyacrylamide gel electrophoresis.

Mice were left untreated or immunized with 90 μg of p33-VLPs mixed with G6 CpGs (20 nmol). Twelve days later, mice were challenged ip with recombinant vaccinia virus expressing LCMV GP (1×10⁶ pfu) and viral titers were determined in ovaries 5 days later (Storni et al., 2002, J. Immunol. 168: 2880) (FIG. 7 B). G6 was not able to significantly induce protective p33-specific CTL responses.

### Example 8

G6 in Liposomes is Able to Enhance p33-Specific Immunity

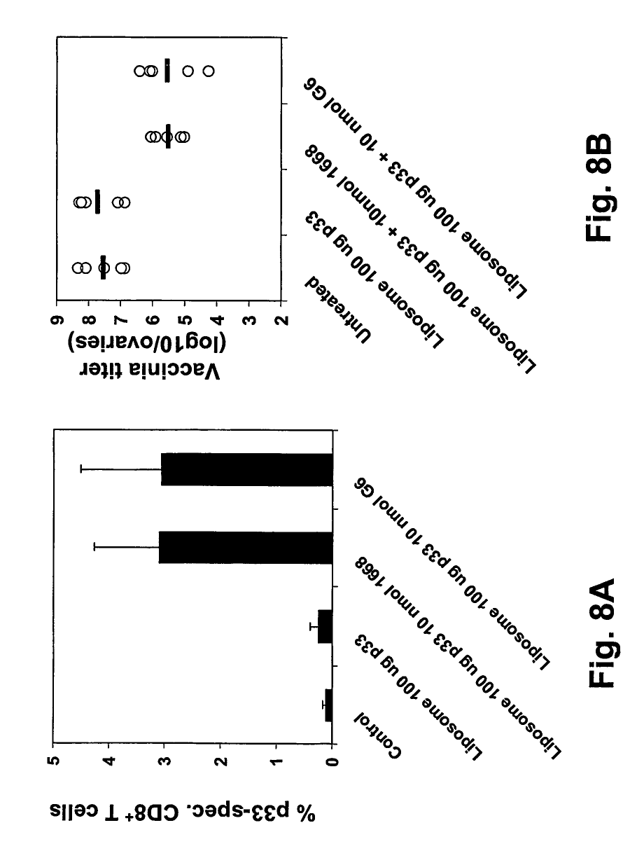

In order to test whether incorporation into liposomes may enhance the efficiency of G6, liposomes containing p33 and either G6 or 1668 were generated. Liposomes were produced as previously described (Ludewig et al, 2000, Vaccine 19, 23-32). Briefly, small unilamellar liposomes were generated by freeze-thawing followed by sequential filter extrusion. The liposomal composition was 200 mg/ml soy phosphatidylcholine, 25 mg/ml cholesterol and 1.2 mg/ml DL-α-tocopherol. The dried lipid mixture was solubilized with 1 mg/ml p33 peptide (KAVYNFATM, SEQ ID NO: 13) alone or with 100 nmol/ml CpGs (G6 or 1668), subjected to 3-5 freeze-thaw cycles and repeatedly extruded through Nucleopore filters of 0.8, 0.4 and 0.2 μm pore size (Sterico AG, Dietikon, Switzerland). Unencapsulated peptide and CpGs were removed by dialysis. Liposome size was determined by laser light scattering (Submicron Particle Sizer Model 370, Nicomp, Santa Barbara, USA). Mice were vaccinated subsequently with the liposomes and p33-specific T cell responses were assessed by tetramer-staining 8 days later (FIG. 8A). At day 12, mice were challenged ip with recombinant vaccinia virus expressing LCMV-GP (4×10⁶ pfu) and viral titers were determined in ovaries 5 days later (Storni et al, 2002, J. Immunol. 168: 2880) (FIG. 8B). Using liposomes, both 1668 and G6 were able to enhance protecticive p33-specific CTL responses.

### Example 9

G10 but not 2006 in Liposomes is Able to Enhance Production of IFNα In Vivo

In order to test whether incorporation into liposomes may enhance the ability of G10 or 2006 to trigger the in vivo production of IFNα, liposomes containing p33 and either G10 or 2006 are generated. Liposomes are produced as previously described (Ludewig et al, 2000, Vaccine 19, 23-32). Briefly, small unilamellar liposomes are generated by freeze-thawing followed by sequential filter extrusion. The liposomal composition is 200 mg/ml soy phosphatidylcholine, 25 mg/ml cholesterol and 1.2 mg/ml DL-α-tocopherol. The dried lipid mixture is solubilized with 1 mg/ml or 50 μg/ml p33 peptide (KAVYNFATM, SEQ ID NO: 13) alone or with 100 nmol/ml CpGs (G10 or 2006), subjected to 3-5 freeze-thaw cycles and repeatedly extruded through Nucleopore filters of 0.8, 0.4 and 0.2 μm pore size (Sterico AG, Dietikon, Switzerland). Unencapsulated peptide and CpGs are removed by dialysis. Liposome size is determined by laser light scattering (Submicron Particle Sizer Model 370, Nicomp, Santa Barbara, USA). Mice are vaccinated subsequently with the liposomes and production of IFNα is analyzed 6, 12, 18 and 24 hours later in the blood of vaccinated mice.

1. A composition for enhancing the production of IFNα in an animal comprising:
(a) a liposome;
(b) at least one A-type CpG;
wherein all nucleotides of the A-type CpG oligonucleotide (b) are phosphodiester nucleotides, and further wherein said A-type CpG (b) is bound to said liposome (a).

2. The composition of claim 1, wherein said at least one A-type CpG comprises poly G motifs at the 5′ and 3′ ends.

3. The composition of claim 2, wherein the G nucleotides of said poly G motifs are phosphodiester nucleotides.

4. The composition of claim 1, wherein said at least one A-type CpG comprises the sequence 5′R₁Y₁—CG-R₂Y₂3′, and wherein R₁, R₂, Y₁, and Y₂ are any nucleotide.

5. The composition of claim 1, wherein said at least one A-type CpG comprises the sequence 5′R₁Y₁CGR₂Y₂3′ or 5′R₁Y₁CGY₂R₂3′ wherein R₁, R₂, or R₃ is A or G, and Y₁, Y₂, or Y₃ is C or T.

6. The composition of claim 5, wherein said at least one A-type CpG comprises the sequence 5′R₁R₂CGR₃Y₁CGY₂Y₃3′.

7. The composition of claim 1, wherein said A-type CpG is selected from:
(a) a recombinant oligonucleotide;
(b) a genomic oligonucleotide;
(c) a synthetic oligonucleotide;
(d) a plasmid-derived oligonucleotide;
(e) a PCR product;
(f) a single-stranded oligonucleotide; and
(g) a double-stranded oligonucleotide.

8. The composition of claim 1, wherein said at least one A-type CpG comprises a palindromic sequence.

9. The composition of claim 8, wherein said palindromic sequence consists of GACGATCGTC (SEQ ID NO: 16).

10. The composition of claim 9, wherein said palindromic sequence is flanked at its 5′-terminus by at least 3 and at most 10 guanosine entities and wherein said palindromic sequence is flanked at its 3′-terminus by at least 6 and at most 10 guanosine entities.

11. The composition of claim 10, wherein said A-type CpG has a nucleic acid sequence selected from:

| | |
|---|---|
| (a) GGGGACGATCGTCGGGGGG; | (SEQ ID NO:6) |
| (b) GGGGGACGATCGTCGGGGGG; | (SEQ ID NO:7) |
| (c) GGGGGGACGATCGTCGGGGGG; | (SEQ ID NO:8) |
| (d) GGGGGGGACGATCGTCGGGGGG; | (SEQ ID NO:9) |
| (e) GGGGGGGGACGATCGTCGGGGGGG; | (SEQ ID NO:10) |
| (f) GGGGGGGGGACGATCGTCGGGGGGGG; | (SEQ ID NO:11) |
| (g) GGGGGGGGGGACGATCGTCGGGGGGGGG; | (SEQ ID NO:12) |
| (h) GGGGGGCGACGACGATCGTCGTCGGGGGGG; | (SEQ ID NO:5) |
| and | |
| (i) GGGGGGGGGGGACGATCGTCGGGGGGGGGG. | (SEQ ID NO:3) |

12. The composition of claim 9, wherein said at least one A-type CpG has a nucleic acid sequence of SEQ ID NO: 3.

13. The composition of claim 1, wherein said liposome is selected from the group consisting of:
(a) neutral;
(b) anionic;
(c) cationic;
(d) stealth; and
(e) cationic stealth.

14. The composition of claim 1, wherein said liposome is a cationic liposome.

15. A method for enhancing the production of IFNα in an animal, said method comprising introducing into said animal the composition of claim 1.

16. (canceled)

17. The method of claim 13, wherein said composition is introduced into said animal subcutaneously, intramuscularly, intravenously, intranasally, directly into the lymph node or locally into, onto or close to a tumor.

18-22. (canceled)

23. The composition of claim 1, wherein said A-type CpG comprises 20 to 300 nucleotides.

24. The composition of claim 23, wherein said A-type CpG comprises 20 to 100 nucleotides.

25. The composition of claim 24, wherein said A-type CpG comprises 20 to 40 nucleotides.

26. A method of treatment of a disorder or disease in an animal, wherein said disorder or disease is selected from the group consisting of cancer and infectious diseases, the method comprising introducing the composition of claim 1 into said animal.
